(19) United States
(12) Patent Application Publication
(10) Pub. No.: US 2012/0308578 A1
(43) Pub. Date: Dec. 6, 2012

(54) SLE-DC-MEDIATED ENHANCED IgG- AND IgA-SECRETING B CELL RESPONSES

(75) Inventors: SangKon Oh, Baltimore, MD (US); Maria Virginia Pascual, Dallas, TX (US); Jacques F. Banchereau, Montclair, NJ (US); Christine Morel Coquery, Zion Crossroads, VA (US); HyeMee Joo, Dallas, TX (US)

(73) Assignee: BAYLOR RESEARCH INSTITUTE, Dallas, TX (US)

(21) Appl. No.: 13/489,851

(22) Filed: Jun. 6, 2012

Related U.S. Application Data
(60) Provisional application No. 61/493,755, filed on Jun. 6, 2011.

(51) Int. Cl. G01N 33/566; A61P 19/04; G01N 21/64; A61K 39/395; C12Q 1/68; G01N 27/62

(52) U.S. Cl. 424/158.1; 435/6.11; 436/501; 435/7.21; 435/6.12; 435/6.1; 424/130.1

(57) ABSTRACT

The present invention provides a method for treating a patient at risk for or diagnosed systemic lupus erythematosus (SLE) by determining the overall expression of syndecan-1 in one or more cells of a patient suspected of having SLE; and predicting the efficacy of a therapy with a pharmaceutical agent for treating the patient, wherein a decrease in the overall expression of syndecan-1 in the patient cells when compared to the expression of syndecan-1 in normal cells indicates a predisposition to responsiveness to anti-neoplastic agent therapy, wherein the therapy comprises administering an effective amount of the pharmaceutical agent to the patient.

### CROSS-REFERENCE TO RELATED APPLICATIONS

The present application claims the benefit of priority to U.S. Provisional Patent Application No. 61/493,755 filed Jun. 6, 2011, the entire contents of which are incorporated herein.

### STATEMENT OF FEDERALLY FUNDED RESEARCH

This invention was made with U.S. Government support under Contract Nos. P50-AR05503 National Institutes of Health (NIH). The government has certain rights in this invention.

### TECHNICAL FIELD OF THE INVENTION

The present invention relates in general to the field novel therapeutic targets to treat this disease, and more particularly, to monocyte differentiation into dendritic cells in a type I IFN-dependent manner.

### INCORPORATION-BY-REFERENCE OF MATERIALS FILED ON COMPACT DISC

None.

### REFERENCE TO A SEQUENCE LISTING

The present application includes a Sequence Listing, and is hereby incorporated by reference in its entirety.

### BACKGROUND OF THE INVENTION

Without limiting the scope of the invention, its background is described in connection with biomarkers for systemic lupus erythematosus (SLE).

For example, U.S. Pat. No. 7,713,925 issued to Ekker, et al., entitled, “Syndecans and Angiogenesis” discloses methods and materials related to modulating syndecan levels and angiogenesis in an animal. The invention provides syndecan polypeptides and nucleic acids encoding syndecan polypeptides, including dominant negative syndecan polypeptides. The invention also provides polynucleotides and polynucleotide analogues for modulating angiogenesis, as well as cells and embryos containing the polynucleotides and polynucleotide analogues. The invention further provides methods for identifying syndecan- and angiogenesis-modulating agents.

U.S. Pat. No. 7,598,031 issued to Liew entitled, “Method for the Detection of Gene Transcripts in Blood and Uses Thereof” discloses detection and measurement of gene transcripts in blood. Specifically provided is a RT-PCR analysis performed on a drop of blood for detecting, diagnosing and monitoring diseases using tissue-specific primers. The present invention also describes methods by which delineation of the sequence and/or quantitation of the expression levels of disease-associated genes allows for an immediate and accurate diagnostic/prognostic test for disease or to assess the effect of a particular treatment regimen.

U.S. Pat. No. 7,910,299, issued to Behrens, et al. entitled, “Methods for diagnosing systemic lupus erythematosus,” relating to methods and materials involved in diagnosing SLE. More particularly, the invention relates to methods and materials involved in diagnosing SLE, diagnosing severe SLE, and assessing a mammal's susceptibility to develop severe SLE. For example, the invention provides nucleic acid arrays that can be used to diagnose SLE in a mammal. Such arrays can allow clinicians to diagnose SLE based on a simultaneous determination of the expression levels of many genes that are differentially expressed in SLE patients as compared to healthy controls.

### SUMMARY OF THE INVENTION

In one embodiment, the present invention includes a method for diagnosing systemic lupus erythematosus (SLE) comprising the steps of: determining a syndecan-1 expression level of a sample; comparing the syndecan-1 expression level in the sample to a normal level of expression in a control sample of a known normal tissue; and correlating the syndecan-1 expression level to a diagnoses of systemic lupus erythematosus, wherein an increased syndecan-1 expression level indicates a diagnoses of systemic lupus erythematosus. In one aspect, the sample comprises a tissue sample, a fluid or a supernatant. In another aspect, the sample comprises one or more cells from a patient. In another aspect, the step of determining the syndecan-1 expression level comprises hybridization with an allele specific probe, an antibody probe, or immunohistochemistry. In another aspect, the syndecan-1 expression level is determined by performing mass spectrometry analysis of syndecan-1 nucleic acids obtained from the individual, rolling circle amplification of a portion of a syndecan-1 nucleic acid obtained from the individual, hybridization with an allele specific probe, performing FISH analysis of syndecan-1 nucleic acids obtained from the individual, performing RT-PCR analysis of syndecan-1 nucleic acids obtained from the individual, performing sequencing analysis of syndecan-1 nucleic acids obtained from the individual, hybridization with an antibody probe or immunohistochemistry. In yet another aspect, the control sample is the syndecan-1 expression level obtained at an earlier timepoint.

Another embodiment of the present invention includes a method for monitoring the progression of systemic lupus erythematosus comprising the steps of: providing a sample from a patient having systemic lupus erythematosus; determining a syndecan-1 expression level in the sample; comparing the syndecan-1 expression level in the sample to a normal level of expression in a control sample of a known normal tissue to determine a SLE progression of the systemic lupus erythematosus; and evaluating a treatment based on the SLE progression, wherein a decreased syndecan-1 expression level indicates a regression of systemic lupus erythematosus. In one aspect, the SLE standard is obtained at an earlier time point from the patient. In another aspect, the method further comprises the step of recording an initial syndecan-1 expression level as the SLE standard. In another aspect, the step of determining the subject syndecan-1 expression level comprises hybridization with an allele specific probe, an antibody probe, or immunohistochemistry. In another aspect, the syndecan-1 expression level comprises performing mass spectrometry analysis of syndecan-1 nucleic acids obtained from the individual, rolling circle amplification of a portion of a syndecan-1 nucleic acid obtained from the individual, hybridization with an allele specific probe, performing FISH analysis of syndecan-1 nucleic acids obtained from the individual, performing RT-PCR analysis of syndecan-1 nucleic acids obtained from the individual, performing sequencing analysis of syndecan-1 nucleic acids obtained from the individual, hybridization with an antibody probe or immunohistochemistry.

Yet another embodiment of the present invention, a method for treating a patient at risk for systemic lupus erythematosus (SLE) comprising the steps of: obtaining a sample from a patient at risk for systemic lupus erythematosus; determining a syndecan-1 expression level in the sample; comparing the syndecan-1 expression level in the sample to a normal level of expression in a control sample of a known normal tissue to determine a SLE progression of the systemic lupus erythematosus; correlating the syndecan-1 expression level to a diagnoses of systemic lupus erythematosus; administering an effective amount of a pharmaceutical agent to the patient; obtaining a second sample from a patient; determining a second syndecan-1 expression level in the second sample; and predicting the efficacy of the pharmaceutical agent based on the second syndecan-1 expression level, wherein a decrease in the expression of syndecan-1 indicates a predisposition to responsiveness to the pharmaceutical agent. In one aspect, the syndecan-1 expression level is determined by hybridization with an allele specific probe, an antibody probe, or immunohistochemistry. In another aspect, the syndecan-1 expression level is determined by performing mass spectrometry analysis of syndecan-1, nucleic acids obtained from the individual, rolling circle amplification of a portion of a syndecan-1 nucleic acid obtained from the individual, hybridization with an allele specific probe, performing FISH analysis of syndecan-1 nucleic acids obtained from the individual, performing RT-PCR analysis of syndecan-1 nucleic acids obtained from the individual, performing sequencing analysis of syndecan-1 nucleic acids obtained from the individual, hybridization with an antibody probe or immunohistochemistry.

Yet another embodiment of the present invention is a method for stratifying a patient in a subgroup of a clinical trial of a lupus erythematosus therapy comprising the steps of: obtaining a sample from a patient suspected of having lupus erythematosus; determining a of syndecan-1 expression level in the sample to a normal level of expression in a control sample of a known normal tissue; administering a candidate drug to the patient; obtaining a second sample from a patient; determining a second syndecan-1 expression level in the second sample; and comparing the syndecan-1 expression level to the second syndecan-1 expression level to predict the efficacy of the pharmaceutical agent, wherein a decrease of the syndecan-1 expression level indicates a predisposition to responsiveness to the pharmaceutical agent; and stratifying the patient into a subgroup for a clinical trial. In one aspect, the sample comprises a tissue sample, a fluid or a supernatant. In another aspect, the sample comprises one or more cells. In another aspect, the syndecan-1 expression level is determined by hybridization with an allele specific probe, an antibody probe, or immunohistochemistry.

In yet another embodiment, the present invention is a method of performing a clinical trial to evaluate a candidate drug believed to be useful in treating a disease state associated with syndecan-1 gene expression, the method comprising: a) administering a candidate drug to a first subset of patients having lupus erythematosus; b) administering a placebo to a second subset of the patients having lupus erythematosus; c) obtaining a sample from the members of the first subset and the second subset; d) measuring a syndecan-1 expression level from the sample; e) comparing the syndecan-1 expression level in the sample to a normal level of expression in a control sample of a known normal tissue; f) determining if there is a statistically significant reduction in the expression of syndecan-1, wherein a statistically significant reduction indicates that the candidate drug is useful in treating said disease state; and repeating step a) to f). In one aspect, the syndecan-1 expression level is measured by hybridization with an allele specific probe, an antibody probe, or immunohistochemistry.

Yet another embodiment of the present invention includes a method for modulating systemic lupus erythematosus (SLE) through mediation of a B cell response comprising the steps of: identifying a subject suspected of having SLE; and mediating a B cell response by reducing a BAFF level in the subject and reducing a APRIL level in the subject.

Yet another embodiment of the present invention includes a method for treating systemic lupus erythematosus (SLE) through mediation of a B cell response comprising the steps of: providing one or more anti-BAFF antibodies to reduce a BAFF level in the subject; providing one or more anti-IL-10 antibodies to reduce a BAFF level in the subject; and providing one or more TACI-Fc compositions to reduce a APRIL level in the subject. Yet another embodiment of the present invention includes a method for mediated an IgG response in a subject comprising the steps of: identifying a subject in need of treatment; providing one or more anti-BAFF antibodies; and providing one or more anti-IL-10 antibodies, wherein the IgG level is reduced. Yet another embodiment of the present invention includes a method for mediated an IgA and an IgM response in a subject comprising the steps of: identifying a subject in need of treatment; and providing one or more TACI-Fc, wherein the IgA and the IgM levels are reduced. Yet another embodiment of the present invention includes a method for mediated an IgA response in a subject comprising the steps of: providing one or more BCMA-Fc compounds to decreased an IgA level without significantly altering an IgM level. Yet another embodiment of the present invention includes a method for reducing auto-antibodies comprising the steps of: identifying a subject in need of reducing auto-antibodies; providing one or more anti-BAFF antibodies and/or one or more anti-IL-10 antibodies to reduce a BAFF level in the subject; and providing one or more TACI-Fc compositions to reduce a APRIL level in the subject.

### BRIEF DESCRIPTION OF THE DRAWINGS

For a more complete understanding of the features and advantages of the present invention, reference is now made to the detailed description of the invention along with the accompanying figures and in which:

FIGS. 1A and 1B are images illustrating the phenotype and morphology of IFN-DCs and SLE-DCs.

FIGS. 2A-2E are images illustrating IFN-DCs and SLE-DCs induce naive B cell differentiation into plasmablasts (PBs).

FIGS. 3A-3C are images illustrating SLE-DCs induced enhanced IgG and IgA class switching in naïve B cells.

FIGS. 4A-4D are graphs illustrating SLE-DCs enhanced by CD19⁺IgD⁻CD27⁺ and CD19⁺IgD⁺CD27⁺ B cell responses by promoting IgG- and IgA-PB differentiation

FIGS. 5A-5D are images illustrating SLE-DCs upregulate BCL-_{X}L in B cells and further promote PB survival.

FIGS. 6A-6C are images illustrating blood from SLE patients contains increased numbers of CD20CD38+-IgA+ and CD20CD38+-IgG+ PBs in that express CXCR3, CCR5, CCR6, and CCR10.

FIGS. 7A-7C are graphs illustrating BAFF and APRIL secreted from SLE-DCs contribute to IgG- and IgA-C secreting B cell responses, respectively.

FIGS. 8A-8C are images illustrating IL-10 secreted by SLE-DCs contributes to IgG-, but not IgM- or IgA-, secreting B cell responses.

FIGS. 9A-9E are images illustrating trans-presentation of APRIL by SLE-DCs is the key mechanism for the enhanced IgA-secreting B cell responses.

FIGS. 10A-10F are images of APRIL trans-presented by SLE-DCs contribute to enhanced naïve B cell differentiation and proliferation.

FIGS. 11A-11B are images illustrating soluble APRIL fails to induce class switching in naïve B cells in the absence of DCs.

FIG. 12 is an image illustrating a reduction of IgM and IgA production by transwell culture.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The term “tissue sample” (the term “tissue” is used interchangeably with the term “tissue sample”) should be understood to include any material composed of one or more cells, either individual or in complex with any matrix or in association with any chemical. The definition shall include any biological or organic material and any cellular subportion, product or by-product thereof. The definition of “tissue sample” should be understood to include without limitation sperm, eggs, embryos and blood components. Also included within the definition of “tissue” for purposes of this invention are certain defined acellular structures such as dermal layers of skin that have a cellular origin but are no longer characterized as cellular. The term “stool” as used herein is a clinical term that refers to feces excreted by humans.

The term “gene” as used herein refers to a functional protein, polypeptide or peptide-encoding unit. As will be understood by those in the art, this functional term includes both genomic sequences, cDNA sequences, or fragments or combinations thereof, as well as gene products, including those that may have been altered by the hand of man. Purified genes, nucleic acids, protein and the like are used to refer to these entities when identified and separated from at least one contaminating nucleic acid or protein with which it is ordinarily associated. The term “allele” or “allelic form” refers to an alternative version of a gene encoding the same functional protein but containing differences in nucleotide sequence relative to another version of the same gene.

As used herein, “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., a-enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids,” which comprise naturally-occurring or modified nucleic acid bases attached to a polyamide backbone. Nucleic acids can be either single stranded or double stranded.

The term “hybridization” as used herein refers to the process in which two single-stranded polynucleotides bind non-covalently to form a stable double-stranded polynucleotide; triple-stranded hybridization is also theoretically possible. The resulting (usually) double-stranded polynucleotide is a “hybrid.” The proportion of the population of polynucleotides that forms stable hybrids is referred to herein as the “degree of hybridization.” Hybridizations are usually performed under stringent conditions, for example, at a salt concentration of no more than 1 M and a temperature of at least 25° C. For example, conditions of 5× SSPE (750 mM NaCl, 50 mM NaPhosphate, 5 mM EDTA, pH 7.4) and a temperature of 25-30° C. are suitable for allele-specific probe hybridizations. For stringent conditions, see, for example, Sambrook, Fritsche and Maniatis. “Molecular Cloning A laboratory Manual” 2 nd Ed. Cold Spring Harbor Press (1989) which is hereby incorporated by reference in its entirety for all purposes above.

The term “rolling circle amplification (RCA)” as used herein describes a method of DNA replication and amplification that results in a strand of nucleic acid comprising one or more copies of a sequence that is a complimentary to a sequence of the original circular DNA. This process for amplifying (generating complimentary copies) comprises hybridizing an oligonucleotide primer to the circular target DNA, followed by isothermal cycling (e.g., in the presence of a ligase and a DNA polymerase). A single round of amplification using RCA results in a large amplification of the sequences in the circular target to obtain a high concentration the desired oligonucleotide on a single strand of nucleic acid. Because the desired nucleic acid sequence becomes the predominant sequence (in terms of concentration) in the mixture, it is said to be “RCA amplified”. With RCA, it is possible to amplify a single copy of a particular nucleic acid sequence to a level detectable by several different methodologies (e.g., hybridization with a labeled probe; incorporation of biotinylated primers followed by avidin-enzyme conjugate detection; incorporation of 32 P-labeled deoxynucleotide triphosphates, such as dCTP or dATP, into the amplified segment).

As used herein the term “antibody probe” refers to an antibody that is specific for and binds to any target antigen. Such a target antigen may be a peptide, protein, carbohydrate or any other biopolymer to which an antibody will bind with specificity.

The term “biomarker” as used herein in various embodiments refers to a specific biochemical in the body that has a particular molecular feature to make it useful for diagnosing and measuring the progress of disease or the effects of treatment. For example, common metabolites or biomarkers found in a person's breath, and the respective diagnostic condition of the person providing such metabolite include, but are not limited to, acetaldehyde (source: ethanol, X-threonine; diagnosis: intoxication), acetone (source: acetoacetate; diagnosis: diet/diabetes), ammonia (source: deamination of amino acids; diagnosis: uremia and liver disease), CO (carbon monoxide) (source: CH₂Cl₂, elevated % COHb; diagnosis: indoor air pollution), chloroform (source: halogenated compounds), dichlorobenzene (source: halogenated compounds), diethylamine (source: choline; diagnosis: intestinal bacterial overgrowth), H (hydrogen) (source: intestines; diagnosis: lactose intolerance), isoprene (source: fatty acid; diagnosis: metabolic stress), methanethiol (source: methionine; diagnosis: intestinal bacterial overgrowth), methylethylketone (source: fatty acid; diagnosis: indoor air pollution/diet), O-toluidine (source: carcinoma metabolite; diagnosis: bronchogenic carcinoma), pentane sulfides and sulfides (source: lipid peroxidation; diagnosis: myocardial infarction), H2S (source: metabolism; diagnosis: periodontal disease/ovulation), MeS (source: metabolism; diagnosis: cirrhosis), and Me2S (source: infection; diagnosis: trench mouth).

As used herein the term “immunohistochemistry (IHC)” also known as “immunocytochemistry (ICC)” when applied to cells refers to a tool in diagnostic pathology, wherein panels of monoclonal antibodies can be used in the differential diagnosis of undifferentiated neoplasms (e.g., to distinguish lymphomas, carcinomas, and sarcomas) to reveal markers specific for certain tumor types and other diseases, to diagnose and phenotype malignant lymphomas and to demonstrate the presence of viral antigens, oncoproteins, hormone receptors, and proliferation-associated nuclear proteins.

The term “statistically significant” differences between the groups studied, relates to condition when using the appropriate statistical analysis (e.g. Chi-square test, t-test) the probability of the groups being the same is less than 5%, e.g. p<0.05. In other words, the probability of obtaining the same results on a completely random basis is less than 5 out of 100 attempts.

The term “genotoxic agent” as used herein is defined to include both chemical and physical agents capable of causing damage to human DNA or the gene. Carcinogens and mutagens are common examples of chemical genotoxic agents, while UV radiation, y and X-rays and the like when they produce oxidized DNA product are common examples of physical genotoxic agents.

The term “anti-neoplastic agent” refers to agents that have the functional property of inhibiting the development or progression of a neoplasm in a mammal, e.g., a human, and may also refer to the inhibition of metastasis or metastatic potential.

The term “kit” or “testing kit” denotes combinations of reagents and adjuvants required for an analysis. Although a test kit consists in most cases of several units, one-piece analysis elements are also available, which must likewise be regarded as testing kits.

Systemic lupus erythematosus (SLE) is an autoimmune disease characterized by multi-organ involvement and immunological abnormalities that include aberrant auto-reactive B cell responses. In SLE, elevated levels of auto-antibodies, particularly anti-dsDNA, are considered to be pathogenic as changes in their titer correlate with disease activity (Arbuckle et al., 2003; Kotzin, 1996). Furthermore, autoantibody-derived immune complexes (ICs) deposit in tissues and exacerbate SLE disease pathogenesis (Koffler et al., 1971; Koffler et al., 1967; Mannik et al., 2003).

The present inventors have recognized that expression of mH chain is an important checkpoint in B cell development. In mice deficient for IgM transmembrane tail exons (μMT mice) B cell development is blocked at the pro-B stage. However, we showed that fas-deficient μMT mice (μMT/lpr) develop a very small population of isotype-switched B cells and produce high titers of self-reactive serum antibodies. In addition, μMT/lpr mice develop severe lymphoproliferation and both pathologic processes occur at young ages, suggesting that a lack of Fas-Fas ligand signaling exacerbates murine lupus in B cell lymphopenic mice. μMT/lpr mouse is particularly permissive for the development and accumulation of antibody-producing cells, thereby explaining the high titers of serum antibodies in these mice. The accumulating cells in μMT/lpr mice express the membrane proteoglycan syndecan-1, a known plasma cell marker. Development of these cells is blocked in mice deficient for TCRβ and TCRδ. The present inventors found that both antibody production and lymphoproliferation in mMT/lpr mice are Th1 regulated. These results, therefore, demonstrate that in the μMT/lpr mouse model a small population of isotype-switched B cells is sufficient for the initiation and propagation of Th1-regulated murine lupus.

The development of auto-antibodies is a hallmark of systemic lupus erythematosus (SLE). SLE serum induces monocyte differentiation into dendritic cells (DCs) in a type I IFN-dependent manner. Such DCs activate T cells, but whether they contribute to B cell alterations in this disease is not known. Here, we demonstrate that SLE serum-induced monocyte-derived DCs (SLE-DCs) can efficiently stimulate naïve and memory B cell differentiation into IgG- and IgA-plasmablasts (PBs) phenotypically identical to those found expanded in the blood of SLE patients. SLE-DC-mediated IgG-PB differentiation is dependent on B cell-activating factor (BAFF) and IL-10. In contrast, IgA-PB differentiation is dependent on a proliferation-inducing ligand (APRIL), but not BAFF nor IL-10. More importantly, SLE-DCs upregulate the expression of CD138 and trans-present APRIL to B cells, which is the key mechanism responsible for the SLE-DC-mediated IgA responses. This feature of SLE-DCs is not shared by DCs generated with IFNα (IFN-DCs) and thus IFN-DCs are much less efficient at inducing IgA responses. In addition, SLE-DCs induce the expression of BCL-X_{L} in PBs and support their survival, further enhancing IgG and IgA responses. Thus, our study supports that a direct interplay between DCs and B cells might contribute to enhance pathogenic antibody responses in SLE.

However, the mechanisms underlying the failure to maintain B cell tolerance in SLE remain incompletely characterized. There are multiple checkpoints during B cell development, maturation and activation that have been demonstrated to be defective in murine lupus models (Grimaldi et al., 2002; Grimaldi et al., 2001; Kuo et al., 1999; Santulli-Marotto et al., 2001) as well as in SLE patients (Wardemann et al., 2003; Yurasov et al., 2006; Yurasov et al., 2005).

SLE patient blood is characterized by B cell lymphopenia and alterations in B cell subset composition. Thus, naïve B cells are decreased while the frequency of immature transitional B cells, CD27⁻ memory B cells, plasmablasts (PBs) and plasma cells (PCs) is increased. (Arce et al., 2001; Odendahl et al., 2000; Wei et al., 2007). The mechanisms underlying the expansion of such populations in SLE blood are not well understood.

DCs play an important role in B cell activation (Dubois et al., 1997; Jego et al., 2003) as well as in tolerance). To highlight their dual functionality, constitutive deletion of DCs in a murine model of lupus shows a marked improvement in disease severity (Teichmann et al.) whereas deletion in a non-autoimmune model resulted in autoimmunity (Ohnmacht et al., 2009). Monocytes represent the most abundant circulating pool of macrophage and DC precursors. Blood monocytes from pediatric SLE patients act as DCs, as they are able to induce the proliferation of CD4⁺ T cells (Blanco et al., 2001). Furthermore, exposure of healthy monocytes to SLE serum results in the generation of cells that have DC morphology and function. Such DC-inducing properties of SLE serum was found to be mainly mediated through IFNα (Blanco et al., 2001). However, SLE serum also contains other factors that might potentiate healthy monocyte differentiation into DCs (Gill et al., 2002), and eventually promote auto-reactive B cell responses in SLE.

SLE serum can induce monocyte differentiation into DCs (SLE-DCs) to reproduce the B cell responses that characterize SLE patients. Our data demonstrate that SLE-DCs have multiple and unique capacities to induce naïve and memory B cell differentiation into IgG- and particularly IgA-secreting PBs. Understanding the mechanisms underlying SLE-DCs-mediated B cell responses might provide novel therapeutic targets to treat this disease.

SLE-DCs display a unique phenotype. SLE serum induces healthy monocyte differentiation into DCs in an IFNα-dependent manner (Blanco et al., 2001). Thus, we first compared the phenotype of DCs generated by culturing healthy monocytes with SLE serum versus IFNα and GM-CSF (IFN-DCs) for three days.

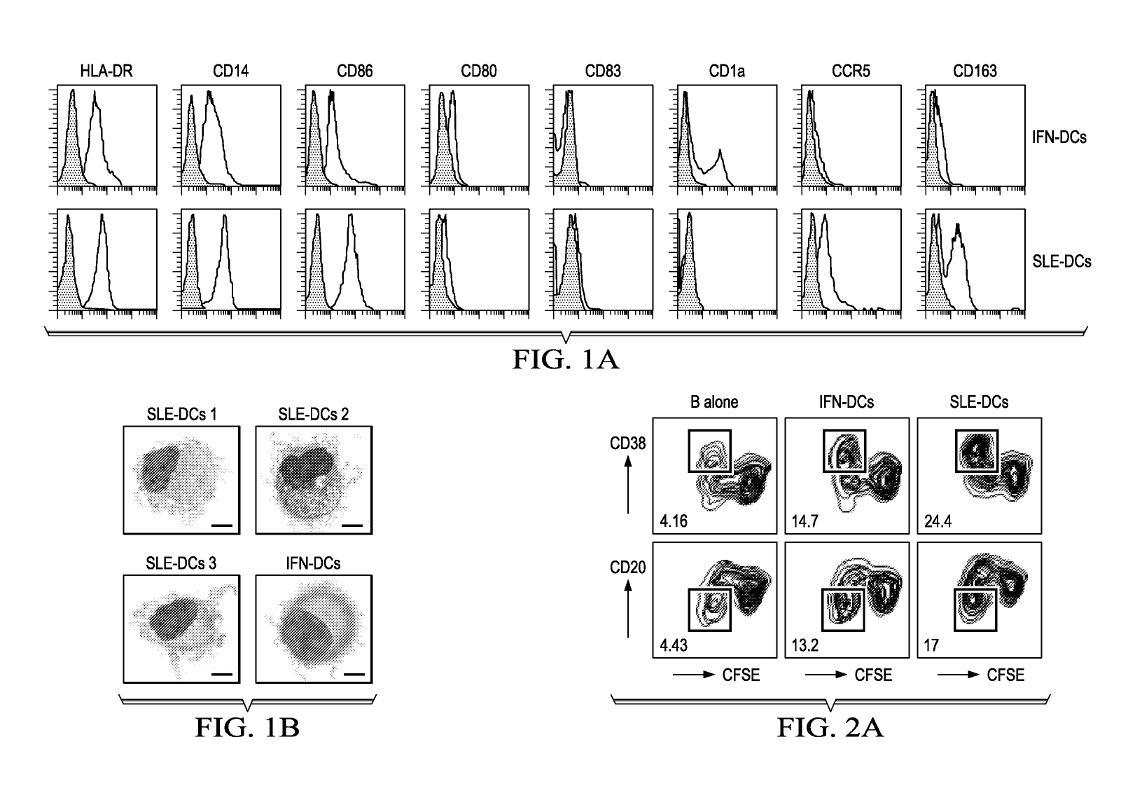

FIGS. 1A and 1B are images illustrating the phenotype and morphology of IFN-DCs and SLE-DCs. FIG. 1A illustrates phenotype of IFN-DCs and SLE-DCs. Solid grey histogram—isotype control, black histogram—marker of interest. FIG. 1B illustrates Giemsa staining of IFN-DCs and SLE-DCs generated with sera from 3 SLE patients. Cells were visualized at 100× magnification. Monocytes from the same healthy donor were used. Representative data is from 10 independent trials using sera from 20 patients and monocytes from 12 healthy donors. Scale bar represents 2.5 μm. Both SLE-DCs and IFN-DCs expressed comparable levels of HLA-DR and CD80, but SLE-DCs expressed higher levels of CD14 and CD86 than IFN-DCs (FIG. 1A). Neither IFN-DCs nor SLE-DCs expressed significant levels of CD83, unless the method to purify monocytes was CD14 positive selection, in which case both SLE-DCs and IFN-DCs expressed CD83 (data not shown), as previously reported (Gill et al., 2002). Most notably, whereas a fraction of SLE-DCs expressed CCR5 and CD163, IFN-DCs expressed only very low levels of CD163, but fractions of IFN-DCs expressed CD1a (FIG. 1A). Cells were further characterized by Giemsa staining, which confirmed that monocytes cultured in SLE serum for three days displayed DC morphology (FIG. 1B). As compared to the fine projections of dendrites found in IFN-DCs, SLE-DCs had thicker dendrites and contained larger numbers of cytoplasmic vacuoles.

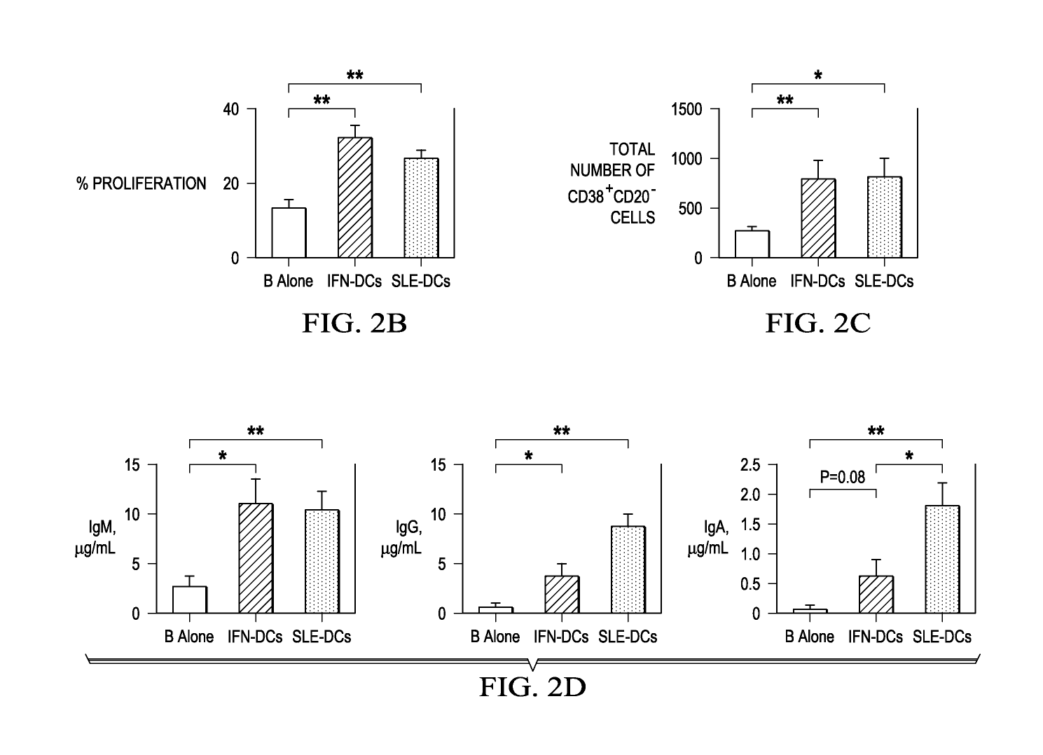

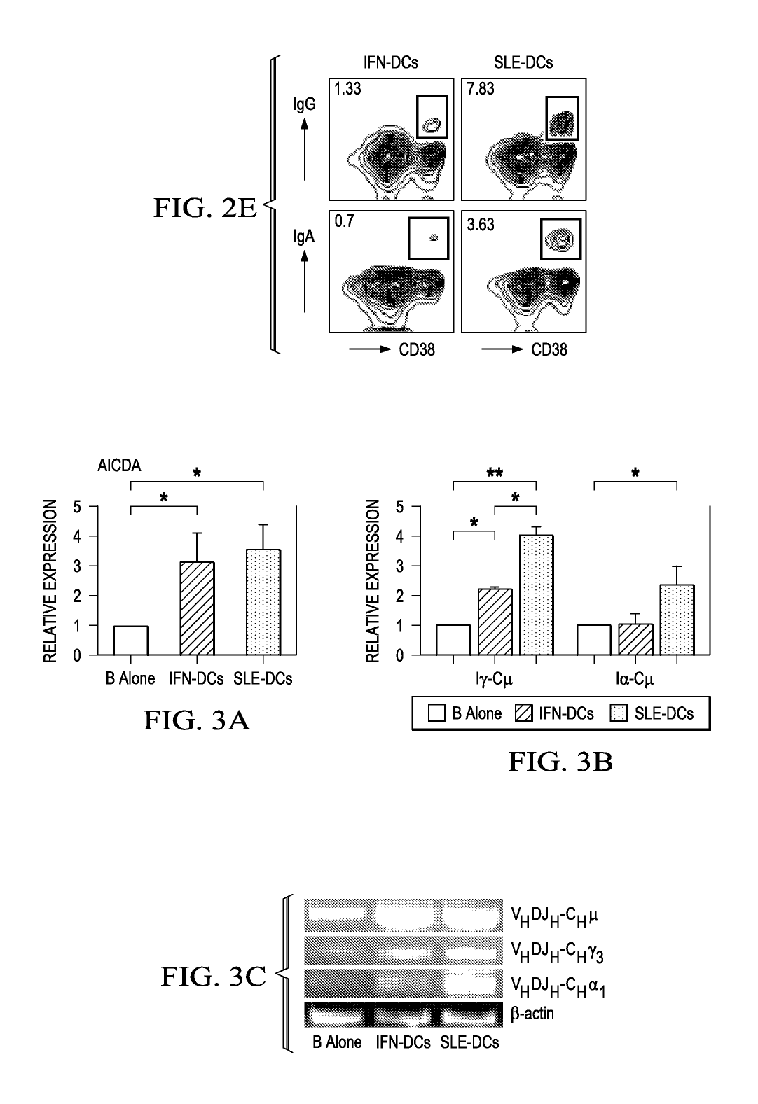

FIGS. 2A-2E are images illustrating IFN-DCs and SLE-DCs induce naive B cell differentiation into plasmablasts (PBs). DCs were co-cultured with CFSE-labeled naïve B cells for 6 or 12 days. (FIG. 2A) CD38 and CD20 expression as well as CFSE-dilution were assessed after 6 days. 1 representative example from 18 separate studies using sera from 36 patients and monocytes from 12 healthy controls. (FIG. 2B) Combined data for Proliferation and (FIG. 2C) PB differentiation. (FIG. 2D) Total immunoglobulin (Ig) assayed by ELISA after 12 days of co-culture. (FIGS. 2B, 2C, 2D) Combined data from 8 separate studies using sera from 16 patients and monocytes/B cells from 6 healthy controls. (FIG. 2E) Intracellular staining of Igs determined after 6 days of co-culture. 1 representative study from 3 independent studies using sera from 6 patients and monocytes/B cells from 4 healthy controls. Statistical significance is denoted as follows: * p<0.05, ** p<0.01. SLE-DCs can efficiently induce naïve B cell differentiation into IgG- and IgA-secreting PBs. To test the roles of SLE-DCs in peripheral B cell responses, purified naïve B cells were co-cultured in the presence or absence of DCs. Both SLE-DCs and IFN-DCs were equally effective in inducing naïve B cell proliferation (FIGS. 2A and 2B) and differentiation into PBs (FIGS. 2A and 2C), as measured by CFSE dilution and CD38 and CD20 expression on B cells, respectively. Interestingly, while IFN-DCs induced high levels of IgM production, their capacity to induce IgG or IgA production was modest (FIG. 2D). SLE-DCs were on the other hand able to induce high levels of IgG and particularly IgA production (FIG. 2D). This was further confirmed by intracellular immunoglobulin (Ig) staining (FIG. 2E).

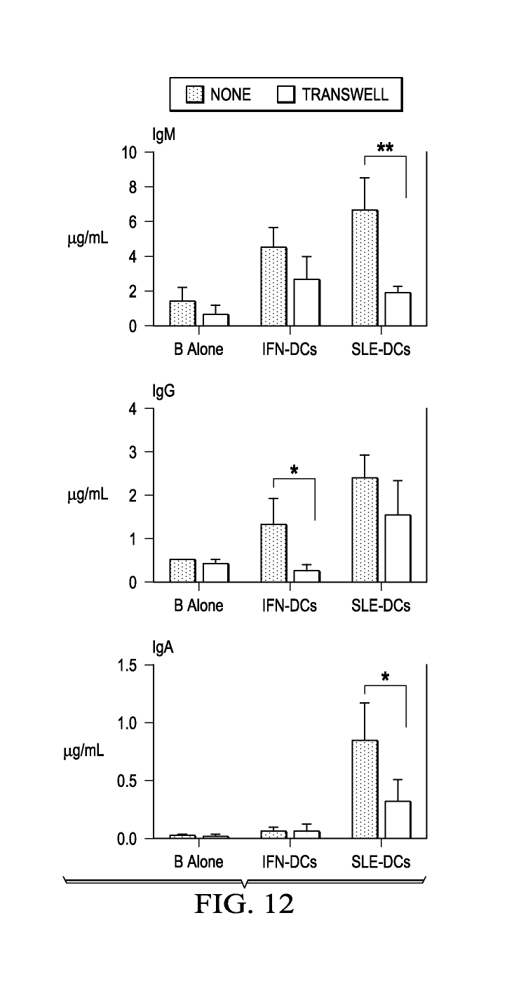

FIGS. 3A-3B are graphs and FIG. 3C is a gel illustrating SLE-DCs induced enhanced IgG and IgA class switching in naïve B cells. CD19⁺IgD⁺CD27⁺ or CD19⁺IgD⁻CD27⁺ B cells were co-cultured with DCs for 6 days or 12 days to assess the level of differentiation and Ig production, respectively. (FIGS. 3A, 3C) Percentage of PBs (CD38⁺CD20⁻) was determined after 6 days of co-culture (FIG. 3B). The amount of total Ig was assayed by ELISA after 12 days of co-culture. Combined data from 3 separate studies using sera from 8 patients (FIGS. 3A, 3C) or 12 patients (FIG. 3B) and monocytes/B cells from 6 healthy controls. Statistical significance is denoted as follows: * p<0.05, ** p<0.01. The lower capacity of IFN-DCs to promote IgG- and IgA-secreting B cell responses were not due to a defect in activation-induced deaminase (AID) expression, as AICDA expression levels were similar in B cells co-cultured with either IFN-DCs or SLE-DCs (FIG. 3A). RT-PCR analysis of switch circles showed that naïve B cells co-cultured with either IFN-DCs or SLE-DCs showed significantly increased levels of Iγ-Cμ (FIG. 3B). However, naïve B cells co-cultured with SLE-DCs, but not IFN-DCs, showed increased levels of IgA circle Iα-Cμ transcripts, demonstrating that SLE-DCs are more efficient than IFN-DCs at inducing class-switching toward both IgG and IgA. Additionally, B cells co-cultured with SLE-DCs showed higher levels of the mature transcripts, V_{H}DJ_{H}-C_{H}γ₃ and V_{H}DJ_{H}-C_{H}α₁, than B cells co-cultured with IFN-DCs. Compared to B cells alone, IFN-DCs also resulted in an increased expression of V_{H}DJ_{H}-C_{H}μ and V_{H}DJ_{H}-C_{H}γ₃ (FIG. 3C). Taken together, these data demonstrate that SLE-DCs possess a unique capacity for enhancing naïve B cell differentiation into IgG- and particularly IgA-secreting PBs.

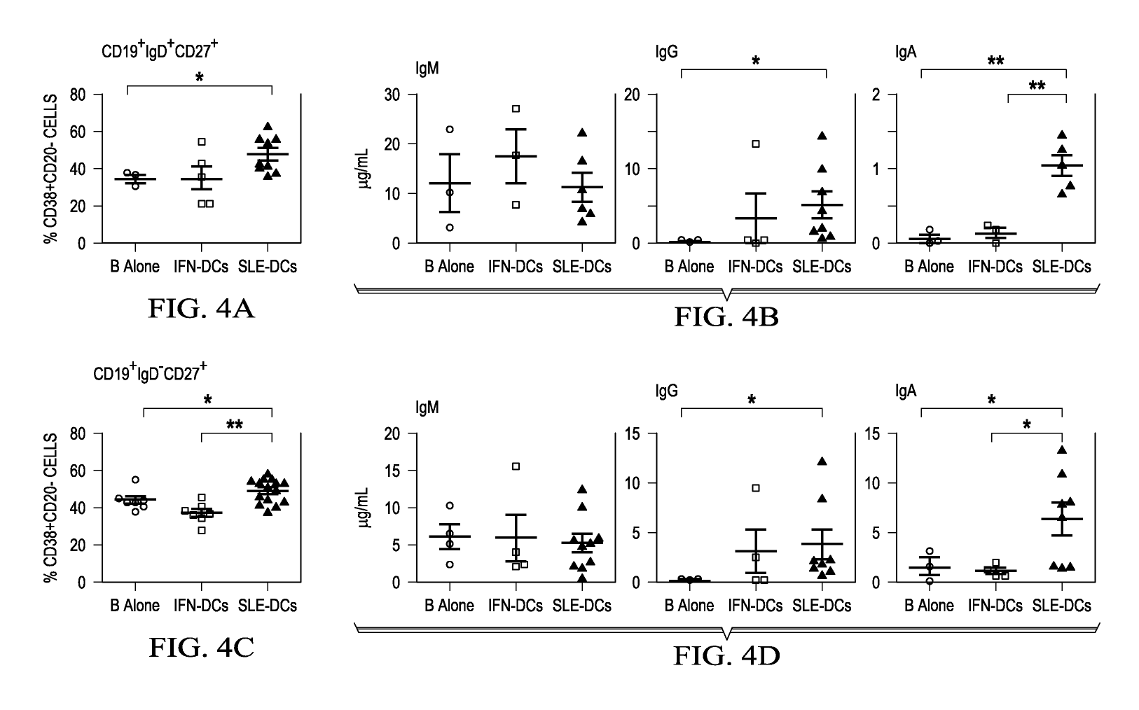

FIGS. 4A-4D are graphs illustrating SLE-DCs enhanced by CD19⁺IgD⁻CD27⁺ and CD19⁺IgD⁺CD27⁺ B cell responses by promoting IgG- and IgA-PB differentiation CD19⁺IgD⁺CD27⁺ or CD19⁺IgD⁻CD27⁺ B cells were co-cultured with DCs for 6 days or 12 days to assess the level of differentiation and Ig production, respectively. (FIGS. 4A, 4C) Percentage of PBs (CD38⁺CD20⁻) was determined after 6 days of co-culture. (FIGS. 4B, 4D, each include 3 separate graphs for IgM, IgG and IgA as noted) The amount of total Ig was assayed by ELISA after 12 days of co-culture. Combined data from 3 separate studies using sera from 8 patients (FIGS. 4A, 4C) or 12 patients (FIGS. 4B, 4D) and monocytes/B cells from 6 healthy controls. Statistical significance is denoted as follows: * p<0.05, ** p<0.01.

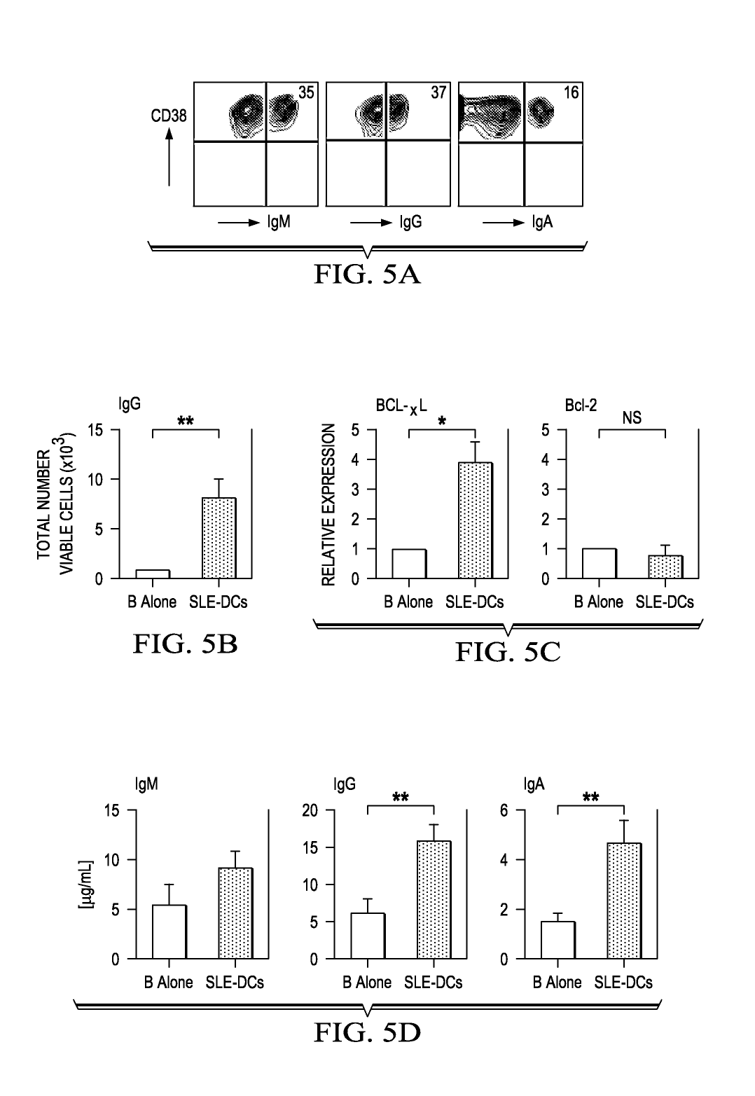

FIGS. 5A-5D (s2) are images illustrating SLE-DCs upregulate BCL-_{X}L in B cells and further promote PB survival. Plasmablasts were expanded from a total B cell population. (FIG. 5A) Surface and intracellular staining of Igs on the PBs was analyzed by flow cytometry. 1 representative studies from 4 separate studies using 8 healthy donors. FACS-sorted PBs (CD19^{low}CD20⁻CD38⁺) were co-cultured with SLE-DCs. (FIG. 5B) The viability of PBs was measured after 6 days of co-culture, using both Trypan Blue staining and 7-ADD by flow cyometry. Combined data from 4 separate studies using sera from 10 patients and monocytes/B cells from 6 healthy donors. (FIG. 5C) RNA was harvested after 2 days. Relative expression of BCL-_{X}L and Bcl-2 was determined by RT-PCR. Combined data from 3 separate studies using sera from 6 patients and monocytes/B cells from 5 healthy controls. (FIG. 5D) Total Igs were measured after 4 days of co-culture of SLE-DCs and PBs by ELISA. Combined data from 4 separate studies using sera from 9 patients and monocytes/B cells from 6 healthy controls. Statistical significance is denoted as follows: * p<0.05, ** p<0.01, NS—not significant.

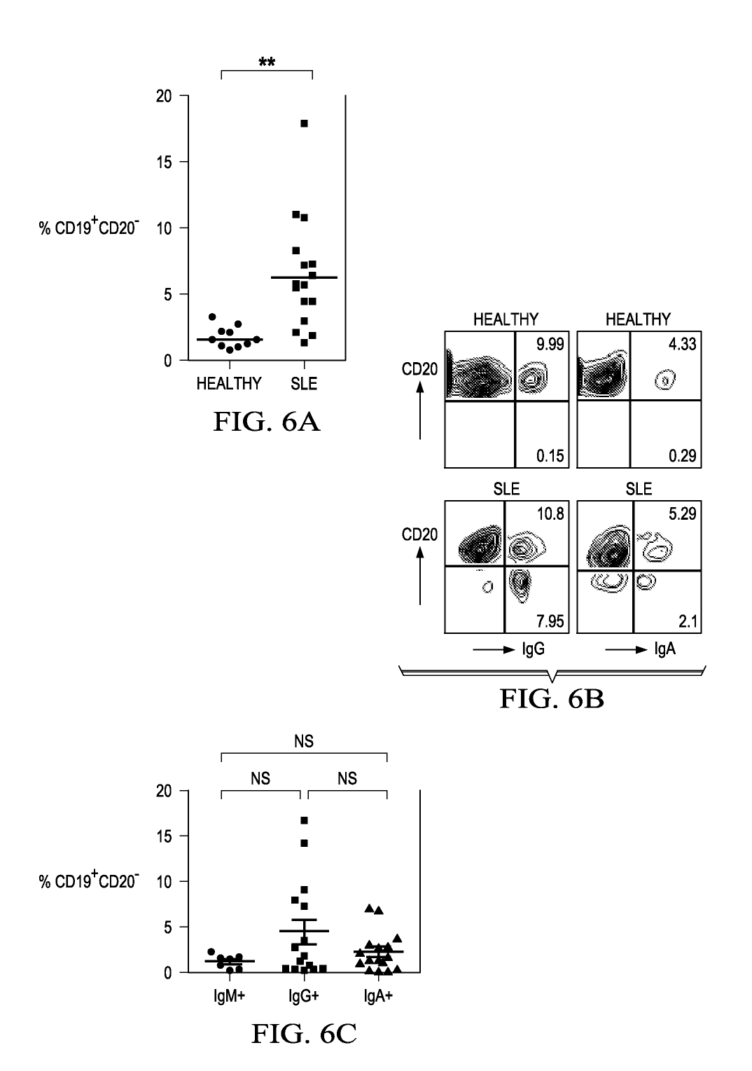

FIGS. 6A-6C are images illustrating blood from SLE patients contains increased numbers of CD20CD38+⁻IgA⁺ and CD20CD38+⁻IgG⁺ PBs. PBMCs were isolated from healthy controls and SLE patients (FIG. 6A) Percentage of CD19⁺CD20⁻ cells was assessed by flow cytometry. Combined data from 10 healthy controls and 16 SLE patients. (FIG. 6B) Surface Ig on the CD20+/− population. Cells were gated initially on CD19⁺7-AAD⁻ population. Representative data from 1 healthy donor and 2 SLE patients from a total of 10 healthy controls and 16 SLE patients tested. (FIG. 6C) Combined data from 16 SLE patients. Statistical significance is denoted as follows: ** p<0.01, NS—not significant.

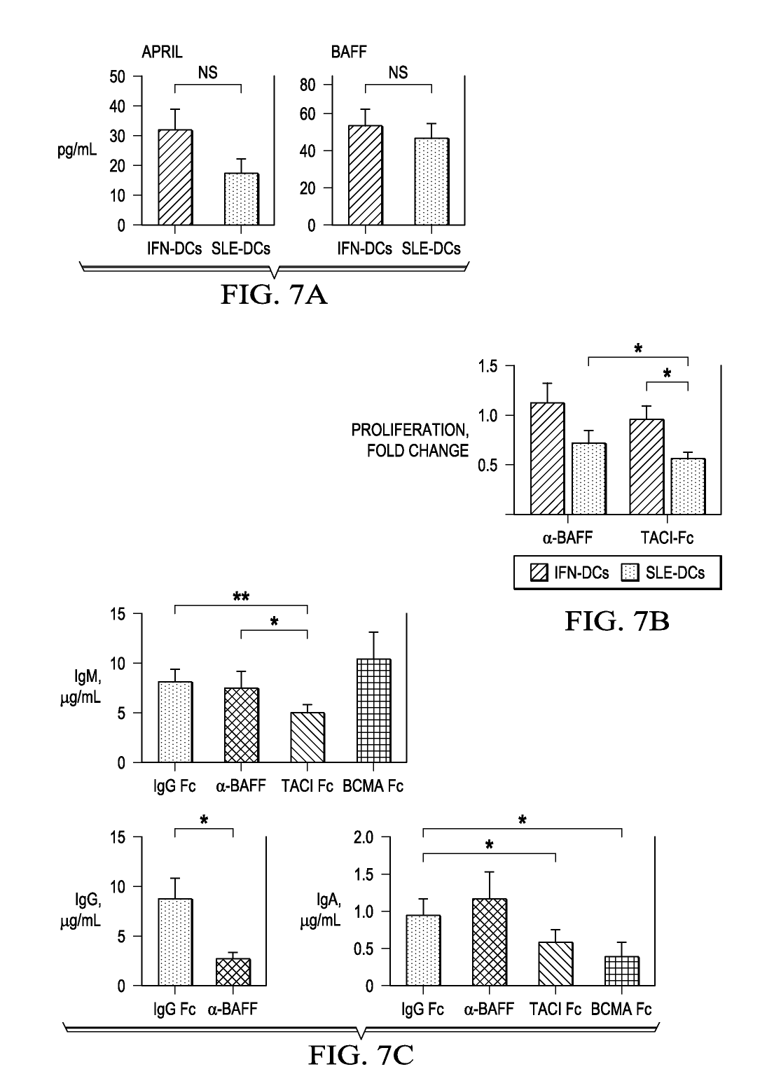

FIGS. 7A-7C are images illustrating BAFF and APRIL secreted from SLE-DCs contribute to IgG- and IgA-secreting B cell responses, respectively. (FIG. 7A) Production of BAFF and APRIL was measured by ELISA after 24 hour culture in serum free media. Combined data from 3 separate studies using sera from 6 SLE patients and monocytes from 6 healthy controls. IFN-DCs and SLE-DCs were co-cultured with naïve sorted B cells for 6 or 12 days in the presence of 10 μg/mL IgG1 control Ab, α-BAFF Ab, TACI or BCMA fusion protein. (FIG. 7B) Proliferation was measured after 6 days gating on the CFSE^{low} population and fold change was compared to IgG control. (FIG. 7C) Ig production was detected by ELISA in harvested supernatant from the SLE-DCB cell co-culture after 12 days. (FIGS. 7B-7C) Combined data from 4 separate studies using sera from 8 SLE patients and monocytes/B cells from 7 healthy controls. Statistical significance is denoted as follows: ** p<0.01, NS—not significant.

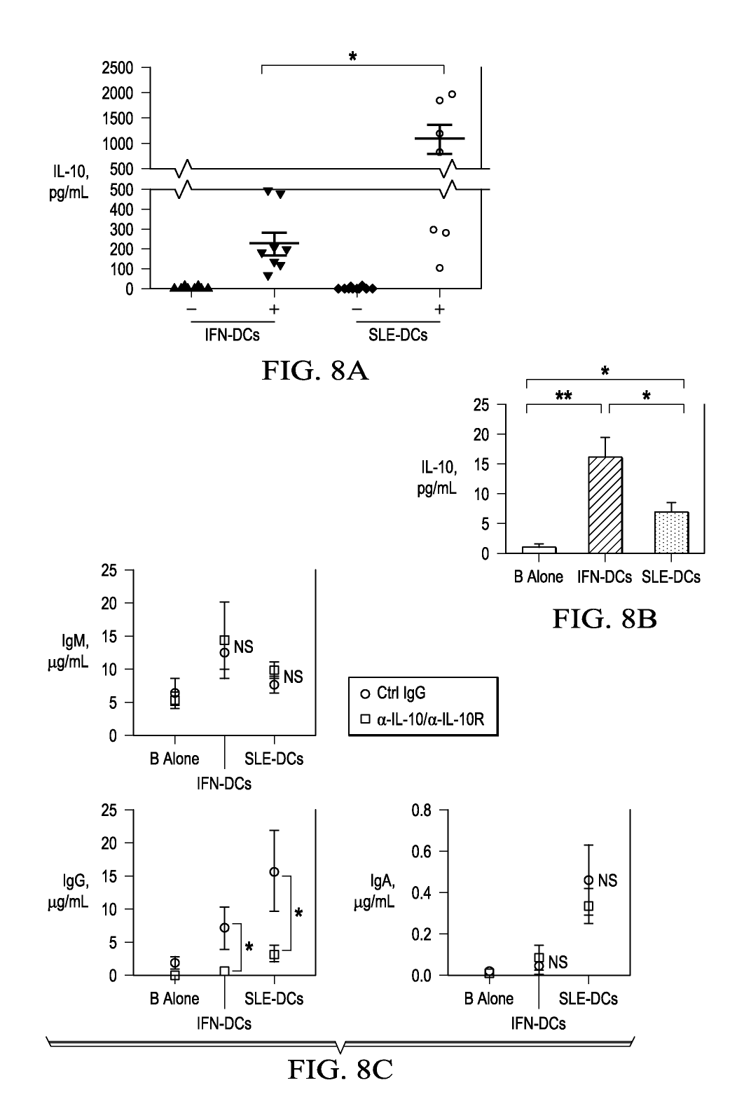

FIGS. 8A-8C are images illustrating IL-10 secreted by SLE-DCs contributes to IgG-, but not IgM- or IgA-, secreting B cell responses. (FIG. 8A) IFN-DCs and SLE-DCs were stimulated in the absence (−) or presence (+) of TLR4L (e. coli LPS) for 24 hours. IL-10 production was measured with a Luminex based platform. (FIG. 8B) IFN-DCs and SLE-DCs were co-cultured with naïve sorted B cells. IL-10 levels were quantified as in (A) after 2 days of co-culture. Combined data from 2 separate studies using sera from 4 SLE patients and monocytes/B cells from 4 healthy controls. (FIG. 8C) IFN-DCs and SLE-DCs were co-cultured with naïve sorted B cells in the presence of 10 μg/mL control IgG (red) or anti-IL-10/anti-IL-10R (blue) for 12 days. Ig production was measured by ELISA. Combined data from 3 studies using sera from 6 SLE patients and monocytes/B cells from 5 healthy controls. Statistical significance is denoted as follows: *—P<0.05, **—P<0.01, NS—not significant. E. coli lipopolysaccharide (LPS)-stimulated SLE-DCs secreted higher levels of both IL-10 (FIG. 8) and IL-8 (data not shown). Furthermore, culture supernatants from the co-culture of naïve B cells and the two DC subsets contain IL-10 (FIG. 8B). Thus we tested the roles of IL-10 in the two types of DC-mediated B cell responses. Addition of neutralizing anti-IL-10 and anti-IL-10R in B cell alone did not alter antibody-secreting B cell responses (FIG. 8C). However, IL-10 neutralization resulted in decreased IgG levels in the co-cultures of naïve B cells and the two subsets of DCs, indicating that IL-10 secreted from both IFN-DCs and SLE-DCs contribute to the enhanced IgG-secreting PB differentiation. Taken together, SLE-DC-mediated enhanced IgG-secreting B cell responses were mainly dependent on BAFF and IL-10, while IgM- and IgA-secreting B cell responses were only partially (≈50%) dependent on either APRIL or both APRIL and BAFF. Combinations of anti-BAFF and TACI-Fc or anti-BAFF and BCMA-Fc did not result in any synergistic effect on the levels of IgA secreted (data not shown).

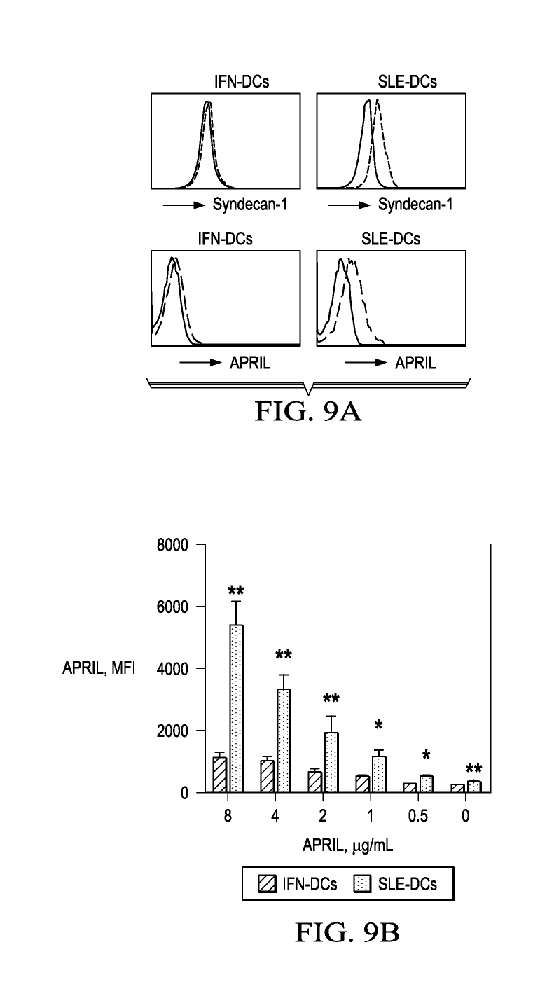

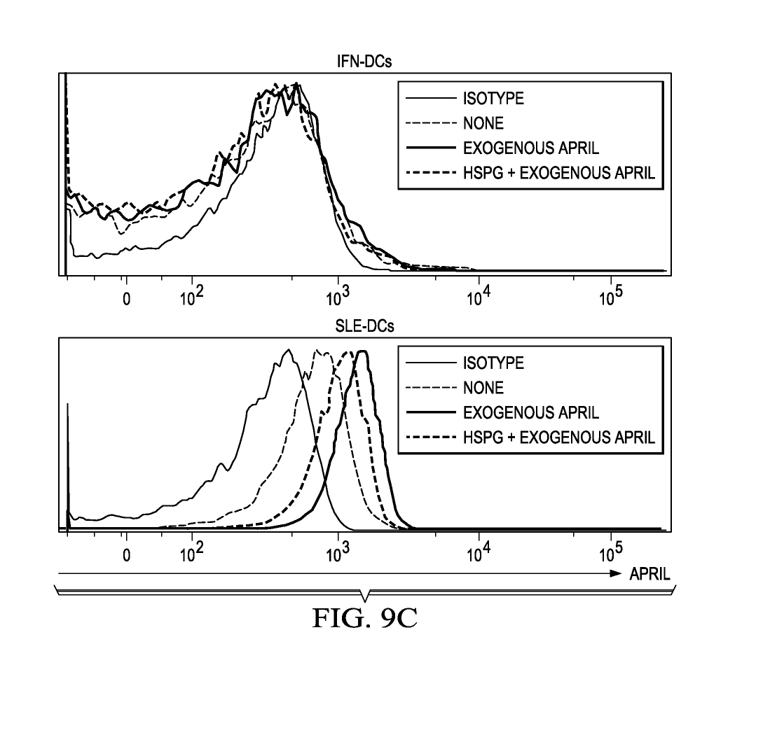

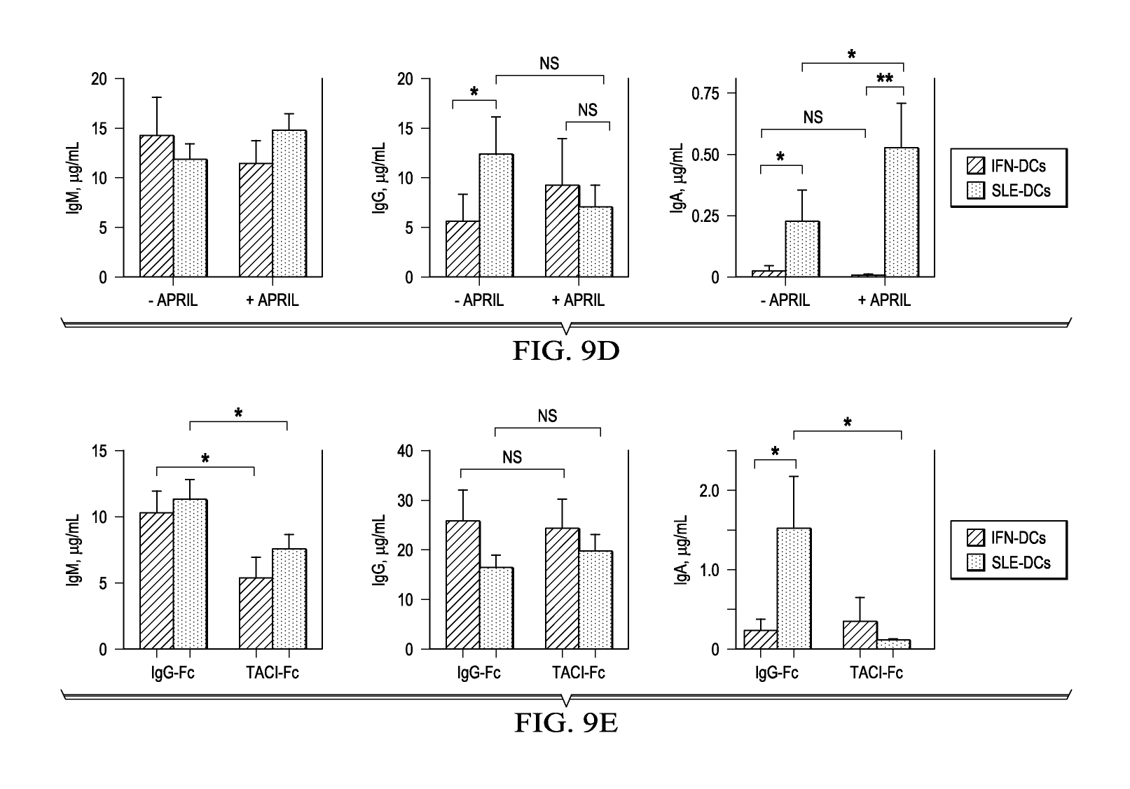

FIGS. 9A-9E (7) Trans-presentation of APRIL by SLE-DCs is the key mechanism for the enhanced IgA-secreting B cell responses. (FIG. 9A) Expression of Syndecan-1 (upper panel) and APRIL (lower panel) on the surface of IFN-DCs and SLE-DCs (black histogram—isotype control, colored histogram—marker of interest). One representative study from 10 separate studies using sera from 24 SLE patients and monocytes from 15 healthy controls. (FIG. 9B) Exogenous APRIL trimer was added to DCs for 30 minutes. Extent of APRIL binding was determined by flow cytometry. Combined data from 3 separate studies using SLE-DCs made with sera from 8 patients and 4 healthy controls. (FIG. 9C) A competition assay was performed using 100 μg/mL heparin sulfate proteoglycans 2. hours prior to the addition of the APRIL to the DCs. Representative data from 3 separate studies, using sera from 7 SLE patients and monocytes from 3 healthy controls. (FIG. 9D) DCs were either untreated (−APRIL) or pre-incubated (+APRIL) with APRIL trimer, then co-cultured with naïve sorted B cells for 12 days (FIG. 9E) DCs that had been pre-treated with APRIL trimer were incubated with naïve sorted B cells in the presence of 10 μg/mL control IgG or TACI Fc for 12 days. (FIGS. 9D, 9E) Ig was measured by ELISA. Combined data from 5 separate studies using sera from 11 SLE patients and monocytes/B cells from 6 healthy controls. Statistical significance is denoted as follows: *—P<0.05, **—P<0.01, NS—not significant.

Trans-presentation of APRIL by SLE-DCs is the key mechanism for enhanced IgA-secreting B cell responses. We further sought additional mechanisms whereby SLE-DCs could promote IgA-secreting B cell responses. Although APRIL is expressed by DCs and macrophages, other cell types, including neutrophils, are also major sources of APRIL in vivo (reviewed in (Mackay et al., 2003). Recent studies show that APRIL binds to heparin sulfate proteoglycans (HSPGs), such as syndecan-1 (CD138) expressed on PCs (Ingold et al., 2005). In this study, it was found that SLE-DCs expressed high levels of syndecan-1 (CD138) on their surface (NCBI Reference Sequence: NM_{—}001006946 (nucleic acid); NP_{—}001006947 (Protein), both incorporated herein by reference). Compared to SLE-DCs, IFN-DCs expressed minimal levels of syndecan-1. More importantly; SLE-DCs were able to present APRIL on their surface. IFN-DCs expressed only minimal levels of surface APRIL. We could not detect surface BAFF on either SLE-DCs or IFN-DCs (data not shown). In addition, SLE-DCs, but not IFN-DCs, could efficiently capture exogenous APRIL and present it on their surface in a dose-dependent manner. Pre-incubation of APRIL with heparin sulfate proteoglycans (HSPGs) resulted in decreased surface-bound APRIL, which suggests that APRIL binding to the surface of SLE-DCs is largely dependent on syndecan-1 expressed on SLE-DCs. IFN-DCs, which expressed minimal levels of syndecan-1, did not efficiently present APRIL on their surface, and thus the pre-incubation of APRIL with HSPG did not alter APRIL expression on the surface of IFN-DCs. Neither BCMA nor TACI were detected on the surface of SLE-DCs or IFN-DCs (data not shown).

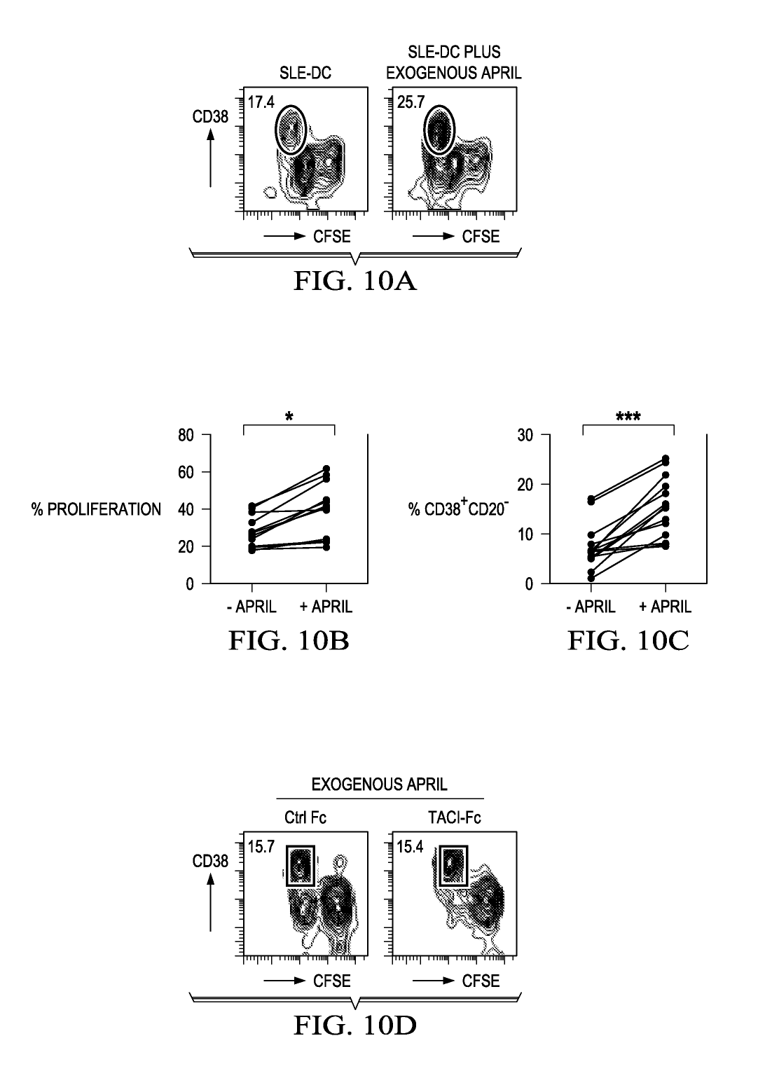

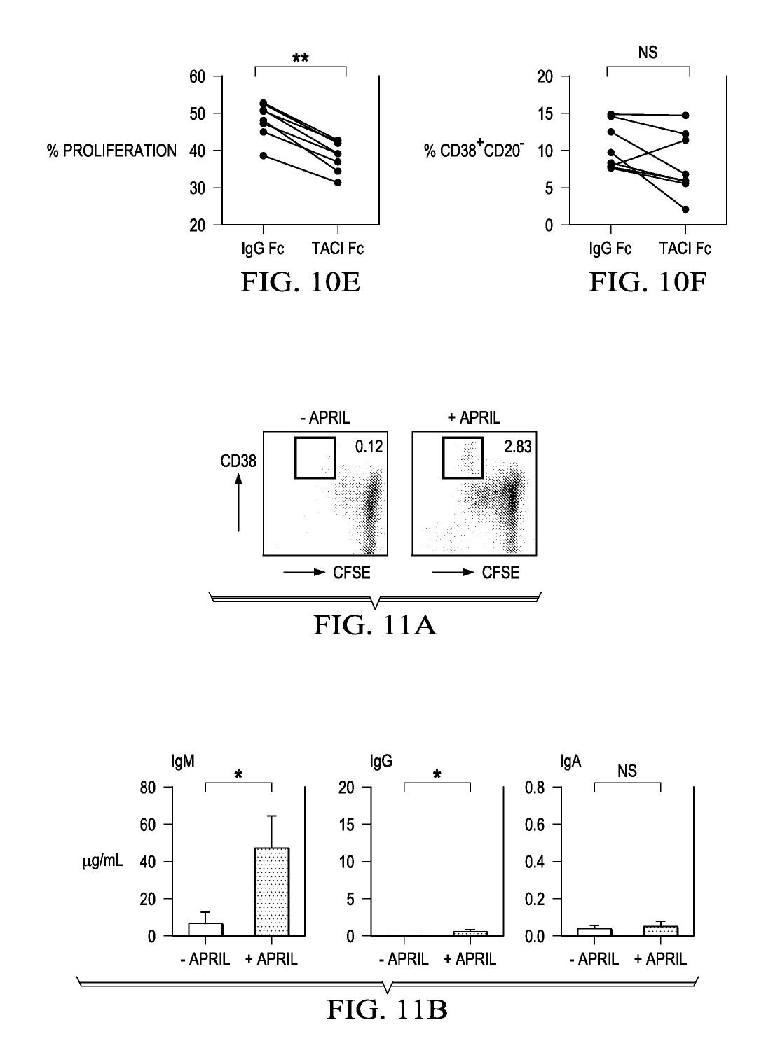

FIGS. 10A-10F are images of APRIL trans-presented by SLE-DCs contribute to enhanced naïve B cell differentiation and proliferation. (FIGS. 10A-C) SLE-DCs were either pre-loaded (+APRIL) or unloaded (−APRIL) with APRIL trimer prior to co-culture with naïve sorted B cells. Differentiation and proliferation were measured after 6 days of co-culture. (FIG. 10A) One representative study. (FIG. 10B, FIG. 10C) Combined data from 6 separate studies using sera from 12 patients and healthy monocytes/B cells from 8 individuals. (FIGS. 10D-F) SLE-DCs were pre-loaded with APRIL trimer and co-cultured with naïve sorted B cells in the presence of either 10 μg/mL control IgG Fc or TACI-Fc. Differentiation and proliferation were measured after 6 days of co-culture. (FIG. 10D) One representative study. (FIGS. 10E, 10F) Combined data from 4 separate studies using sera from 10 patients and healthy monocytes/B cells from 4 individuals. Statistical significance is denoted as follows: *—P<0.05, **—P<0.01, ***—P<0.001, NS—not significant.

To test the roles of membrane-bound APRIL in B cell responses, we incubated both DCs with APRIL for 30 minutes, washed out any residual APRIL and co-cultured the DCs with naïve B cells. While APRIL-fed IFN-DCs showed a modest increase in naïve B cell PB differentiation (data not shown), APRIL-fed SLE-DCs showed an enhanced ability to induce both naïve B cell proliferation as well as differentiation (FIGS. 10A-C). APRIL-fed SLE-DCs led to an enhanced ability to secrete IgA, but not IgG or IgM by naïve B cells. These observations were further confirmed by blocking membrane-bound APRIL with TACI-Fc (FIG. 7E). TACI-Fc was able to neutralize the activity of surface-bound APRIL and almost completely inhibit the IgA-secreting B cell responses. Surprisingly, while TACI Fc decreased proliferation (FIGS. 10D, 10E) and IgA production by naïve B cells, there was minimal effect on the differentiation of these cells (FIGS. 10D, 10F).

FIGS. 11A-11B are images of soluble APRIL fails to induce class switching in naïve B cells in the absence of DCs. Naïve sorted B cells were cultured in the presence of 2 μg/mL of APRIL trimer for 6 or 12 days. (FIG. 11A) Differentiation was determined after 6 days of culture. (FIG. 11B) Ig production was measured after 12 days of culture. Statistical significance is denoted as follows: *—P<0.05, NS—not-significant. Addition of soluble APRIL directly to naïve B cells resulted in modestly increased proliferation, differentiation (FIG. 11A), IgM and to a lesser extent IgG, but not IgA production (FIG. 11B).

FIG. 12 is an image illustrating a reduction of IgM and IgA production by transwell culture. IFN-DCs and SLE-DCs were loaded into the upper chamber of a transwell plate. B cells were added into the lower chamber of the transwell plate and the cells were co-cultured for 12 days. Ig production was quantified by ELISA. Combined data from 2 separate studies using sera from 4 SLE patients and monocytes/B cells from 3 healthy controls. Statistical significance is denoted as follows: *—P<0.05, **—P<0.01, NS—not significant.

SLE-DCs induce memory B cells to differentiate into IgG- and IgA-PBs. We next tested whether SLE-DCs could also promote CD19⁺IgD⁺CD27⁺ and CD19⁺IgD⁻CD27⁺ memory B cell differentiation into PBs. FIG. 4A shows that SLE-DCs were more efficient than IFN-DCs at inducing IgD⁺CD27⁺ B cells to become PBs (CD38⁺CD20⁻) secreting IgG- and particularly IgA, but not IgM (FIG. 4B). SLE-DCs also enhanced PB differentiation from IgD⁻CD27⁺ B cells (FIG. 4C). Similar to IgD⁺CD27⁺ B cells, IgD⁻CD27⁺ memory B cells co-cultured with SLE-DCs secreted increased levels of IgG and IgA (FIG. 4D). Although both subsets of memory B cells proliferated more than naïve B cells in the absence of DCs, SLE-DCs further enhanced their proliferation (data not shown). Taken together, SLE-DCs can promote both naïve and memory B cell responses by enhancing PB differentiation and by inducing class-switching of Ig heavy chains towards IgG and IgA.

Transwell studies further confirmed that cell-cell interaction was important in SLE-DC-mediated IgM- and IgA-secreting B cell responses, but not IgG-secreting B cell responses (FIG. 12). Taken together, these data demonstrates that trans-presentation of APRIL by SLE-DCs is a key mechanism for SLE-DC-mediated IgA-secreting B cell responses.

The auto-reactive B cell response remains an important clinical feature of SLE and as such, is one of the main targets for lupus treatment. Our study demonstrates for the first time that SLEserum induces monocyte differentiation into DCs with a unique capacity to promote B cell responses, particularly towards IgG- and IgA-PBs, reminiscent of those found expanded in the blood of SLE patients. IFN-DCs, DCs generated with IFNα, lack this capacity.

IFNα in SLE serum is one of the major factors contributing to the differentiation of monocytes into DCs (Blanco et al., 2001). However, IFN-DCs are phenotypically and functionally distinct from SLE-DCs. Thus, a significant fraction of SLE-DCs expresses CCR5 and CD163. CCR5 has been shown to be important in disease progression in lupus nephropathy as well as other glomerular diseases and can be found elevated on the surface of DCs in autoimmune disorders (Furuichi et al., 2000; Pashenkov et al., 2002; Stasikowska et al., 2007). CCR5⁺ monocytes are also found in the renal interstitium of SLE patients (Furuichi et al., 2000; Stasikowska et al., 2007). Not surprisingly, several studies have shown elevated levels of RANTES and MIP-1α in the kidneys and serum of patients with SLE (Lit et al., 2006; Stasikowska et al., 2007; Vila et al., 2007). Taken together, our data suggest that SLE serum represents a unique microenvironment for the generation of DCs that could play a role in disease pathogenesis.

As SLE serum induces monocytes to become DCs able to activate T cells, we next asked whether SLE DCs might contribute to enhance B cell responses. Interestingly, while equally adept at inducing proliferation and generating total PBs, SLE-DCs were more efficient than IFN-DCs at generating IgG- and particularly IgA-PBs from naïve B cells. This finding was consistent with the superior capacity of SLE-DCs to induce class-switching toward these isotypes. SLE-DC-mediated IgG- and IgA-secreting B cell responses were not limited to naïve B cells, but extended to CD19⁺IgD⁺CD27⁺, CD19⁺IgD⁻CD27⁺ B cell subsets. Our data suggest that IFNα is not the sole factor in SLE serum promoting B cell responses by acting on monocytes. Indeed, while BAFF is known to be IFN-inducible, we found that SLE serum upregulates APRIL expression on monocytes in an IFN-independent manner (Patel et al., manuscript in preparation). The pathogenic role of IgG auto-antibodies is relatively well understood, but that of IgA is largely unknown, especially in the context of SLE. This might stem from the relative proportion of the two subclasses that can be found in tissue and serum (70-75% IgG, 15% IgA). Some reports have described a profound increase in the serum levels of IgA in lupus patients compared to controls (Conley and Koopman, 1983). Of interest, patient IgA had a lower degree of glycosylation compared to controls, which has been linked to the pathogenic nature of this isotype in a related disease, IgA Nephropathy (Donadio et al., 1978; Matei and Matei, 2000). In addition, deposits of IgA are found in the kidneys of patients with SLE (Florquin et al., 2001). While not currently used as an inclusion criteria for SLE, IgA auto-Abs such as anti-cardiolipin IgA and anti-β2-glycoprotein-I have been shown to predict disease manifestations such as pro-thrombotic events (Kumar et al., 2009; Mehrani and Petri, 2010; Sweiss et al., 2010; Wilson et al., 1998). In addition, B6.Sle1Sle3 mice produce higher levels of anti-nuclear IgA auto-Abs, which are implicated in IgA nephropathy (Liu et al., 2007). Lupus nephritis is diagnosed clinically in approximately 50% of SLE patients (Donadio and Grande, 2002; Floege and Feehally, 2000). Thus, these findings link the expanded IgA-PB population in the blood of SLE patients with the unique capacity of SLE-DCs to generate IgA-switching and PB differentiation.

The capacity of SLE-DCs to induce IgA B cell responses was tracked down to the expression of high levels of surface CD138, which enables the trans-presentation of APRIL to B cells. CD138 was previously described as an APRIL-binding partner, which is the prerequisite for the triggering of TACI- and/or BCMA-mediated B cell activation (Ingold et al., 2005). Another study (Huard et al., 2008) also showed that HSPG-bound APRIL creates unique niches that support the accumulation of IgG- and IgA-PCs in mucosal surfaces. The fact that HSPGs were unable to completely block exogenous APRIL from binding to SLE-DCs suggests that other surface receptors expressed on SLE-DCs might exhibit similar properties. Most importantly, APRIL trans-presented by SLE-DCs was particularly efficient at enhancing IgA-secreting B cell responses, whereas soluble APRIL directly added to B cells only resulted in enhanced IgM-secreting B cell responses, supporting that trans-presentation enhances the mechanism of action of APRIL perhaps as a switching factor.

SLE-DCs secrete not only APRIL, but also BAFF. The contribution of BAFF to B cell-mediated autoimmune diseases is well known. Excessive BAFF expression leads to autoimmunity in mice (Gross et al., 2000; Mackay et al., 1999) and elevated levels of BAFF in SLE serum support auto-reactive naïve B cell (Stohl et al., 2003) and PC survival (Schneider, 2005) through BCMA and BAFF-R. Due to the multiple capacities of both BAFF and APRIL in promoting auto-reactive B cell responses, blocking BAFF or BAFF/APRIL is a promising therapeutic approach to treat B cell-mediated autoimmune diseases, and drugs that block BAFF or BAFF/APRIL have indeed progressed to human clinical trials (Dillon et al., 2006; Sanz and Lee, 2010). Although both selective (BAFF alone) and nonselective (BAFF and APRIL) blockers are being tested, it is not yet known whether there is a therapeutic advantage or an increased toxicity of the nonselective antagonists in translational studies in humans. Since plasma cells predominantly express BCMA and TACI that bind to both BAFF and APRIL, these differences could be physiologically important. In this regard, our findings show that the selective blockade of BAFF reduced only IgG, while nonselective blockers (TACI-Fc and BCMA-Fc) decreased also IgA-secreting B cell responses.

The information obtained from this study is particularly relevant to the generation of extrafollicular T-independent (TI) B cell responses (Fagarasan and Honjo, 2000; Litinskiy et al., 2002). (Crispin et al., 2010), and studying the roles of SLE serum and SLE-DCs in TD-B cell responses is warranted. The type of DC-B cell interaction that we herein describe could also, however, be relevant to the expansion and survival of PBs and PCs from a pool of autoreactive memory B cells initially generated in the context of TD responses. It will be important to test whether combination therapies aimed at blocking these interactions could find a place in the SLE armamentarium.

Seventy-two pediatric patients, who met the American College of Rheumatology Revised Criteria for SLE, were enrolled in this study. Disease activity was measured by SLE Disease Activity Index (SLEDAI). Exclusion criteria: IV steroid, Mycophenolate, Methotrexate, more than 10 mg of prednisone and/or more than 200 mg of Plaquenil. Patients were obtained from the Scottish Rite Hospital for Children (Dallas, Tex.) and Children's Medical Center of Dallas (Dallas, Tex.). Thirty-six healthy controls were recruited at the Baylor Institute for Immunology Research (Dallas, Tex.) and the Scottish Rite Hospital for Children. The study was approved by the Institutional Review Board of Baylor Research Institute and informed consent was obtained from all participants. Blood from patients was obtained during routine examinations. Patient sera were prepared with Topical Thrombin (King Pharmaceuticals, TN).

TABLE 1
Patient Demographics.

| | Gender | | Ethnicity^{a} | | | | | | |
|---|---|---|---|---|---|---|---|---|---|
| | Male | Female | C | AS | AA | H | NR | Age Average | SLEDAI Average |
| SLE | 10 (72) | 62 (72) | 7 | 6 | 20 | 35 | 4 | 14.68 (7-18) | 7.37 (0-22) |
| Healthy | 18 (36) | 18 (36) | 19 | 0 | 2 | 4 | 11 | 18.41 (2-39) | NA |

^{a}C—Caucasian (not of Hispanic decent); AS—Asian; AA—African American; H—Hispanic; NR—Not reported/recorded

Peripheral blood mononuclear cells (PBMCs) were isolated using FICOLL-PAQUE™ PLUS gradient (GE Healthcare, NJ). Monocytes were purified using the EasySep Human Monocyte Enrichment Kit (Negative Selection, StemCell Technologies, BC, Canada) to purity >96%. Total B cells were purified using the EasySep Human B Cell Enrichment Kit (Negative selection, StemCell Technologies, BC, Canada) to purity >98%. Subsets of B cells were further sorted by FACSARIAII™ or FACSVANTAGE™ (BD Biosciences, CA) to yield the following populations: Naïve (CD19⁺IgD⁺CD27⁺), Memory (CD19⁺IgD⁻CD27⁺), Marginal Zone-like B cells (CD19⁺IgD⁺CD27⁺), and PBs (CD19^{low}CD20⁻CD38⁺) to purity >97%.

IFN-DCs and IL-4-DCs were generated by culturing healthy monocytes in serum-free CellGenix® media (CellGenix Technologie, IL) supplemented with 50 ng/mL GM-CSF (Baylor Hospital Pharmacy, TX) and 250 units/mL IFN-α (Schering-Plough, NJ) or 50 ng/mL IL-4 (R&D Systems, MN) for 3 or 6 days, respectively. SLE-DCs were generated by culturing healthy monocytes in CELLGENIX® media supplemented with 25% SLE serum for 3 days.

DC phenotype: Anti-CD163-FITC, anti-CD86-PE, anti-CCR5-PE, anti-BCMA (R&D Systems, NJ), anti-syndecan-1-PE, anti-CD11c-PE, anti-CD1a-FITC, anti-CD80-PE (eBiosciences, CA), and anti-CD14-PerCp and anti-CD83-APC (BioLegend, CA). Purified anti-human APRIL (Alexis Biochemicals, PA) and anti-BCMA Abs (R&D Systems) were conjugated to Alexa-647 using an ALEXA FLUOR® labeling kit (Invitrogen, MOLECULAR PROBES™, CA) Live/dead-aqua (Invitrogen).

B Cell phenotype: Anti-CD38-PECy7 (BioLegend), anti-human IgG-PE, anti-human IgA-APC (Miltenyi, Germany), anti-CD20-PECy5, anti-CD27-APCCy7 (BD Biosciences), CFSE, Live/dead-aqua (Invitrogen), anti-CD138-PE (eBiosciences).

B Cell sorting: Anti-human IgD-PE, anti-CD27-FITC (Southern Biotech, TX), anti-CD19-APC (BD Bioscience), and anti-CD3-Quantum Red (Sigma, MO), anti-CD38-PECy7 (eBiosciences) BD CYTOFIX/CYTOPERM™ and BD PERM/WASH™ (BD Biosciences) were used for intracellular staining as per the manufacturer's instructions. Cells were acquired on a LSRII™ or FACSCANTOII™ (BD Biosciences) and analysis was performed using FlowJo software (Tree Star Inc, OR).

Sandwich ELISAs were performed to measure total IgM, IgG, IgA, APRIL and BAFF. Capture and detection antibodies for Ig ELISAs were purchased from Southern Biotech (TX). Human reference serum (Bethyl, TX) was used to generate the standard curve for immunoglobulin (Ig) ELISAs. APRIL and BAFF ELISAs were performed using reagents provided by ZymoGenetics (WA). Detection of ssDNA and dsDNA utilized the protocol described above except the capture Ag was 10 μg/mL calf thymus (ssDNA or dsDNA) (Sigma) (Mukundan et al., 2009). The presence of ANA was measured using an ELISA Kit (Alpha Diagnostics, TX) as per the manufacturer's instructions, as was the presence of anti-cardiolipin (Calbiotech, CA). All ELISAs utilized Nunc Maxisorp™ plates (Thermo Scientific, MA). Plates were read with a SpectraMax M2 (Molecular Devices, CA) and analyzed with SoftMaxPro V5 software (Molecular Devices).

Cells were stained using the DIFFQUICK™ Stain Set (Siemens, IL) as per the manufacturer's instructions and visualized on an Olympus BX60 microscope at 100× magnification using a Nikon Digital Camera (DXM1200C) and Nikon NIS Elements Software.

4×10⁴ purified B cells were co-cultured with 5×10³ DCs in RPMI medium (Invitrogen) supplemented with HEPES (Invitrogen), non-essential amino acids, L-glutamate (Sigma) Pen/strep and 10% FBS (HyClone, Fisher Bioscience, UT), 20 units/mL IL-2 (R&D Systems), CpG (ODN2006) (Invivogen, CA). 10 μg/mL α-BAFF Ab, (R&D Systems), TACI-Fc & BCMA-Fc (Zymogenetics and R&D systems) and Control IgG₁ (Sigma) were added into B/DC co-culture systems. In some studies, DCs were pre-incubated with 2 μg/mL APRIL trimer (ZymoGenetics) for 30 minutes, then washed before their addition to the B cell co-culture. On day 6, B cells were stained for phenotype and intracellular immunoglobulins (Igs) and culture supernatants were harvested on day 12 for measuring secreted Igs. To generate PBs, purified total B cells were co-cultured with IL-4-DCs for 6 days. CD19⁺CD38⁺CD20⁻ cells were sorted and used as PBs. 4×10⁴ PBs were co-cultured with 5×10³ DCs. For transwell studies, 4×10⁴ purified B cells were seeded in the lower compartment of a 96 well Nunc transwell insert and 5×10³ DCs were added to the upper portion. For some studies, IL-10 and the IL-10 receptor were blocked using reagents from BD (Clone JES3-9D7) and BioLegend (Clone 3F9), respectively. In Ag-specific B cell responses, IFN-DCs and SLE-DCs were pre-incubated with supernatant from MDCK cells infected with Influenza A (PR8) for 30 minutes, then washed prior to co-culture with B cells.

Total RNA was isolated from B cells co-cultured with DCs using the RNAqueous® Micro Kit (Ambion, TX) and cDNA was synthesized using the Reverse Transcription System (Promega, WI). cDNA was amplified with the LIGHT CYCLER® SYBR Green Master I Kit (Roche, NJ), according to the manufacturer's instructions and run On a LightCycler®480 (Roche). Primers for switch circles, mature transcripts, AICDA, and β-actin were as previously described (Dullaers et al., 2009; He et al., 2007); BCL-_{X}L-Fwd-5′-AAGCGCTCCTGGCCTTTC-3′ SEQ ID No 1, BCL-_{X}L-Rev-5′-CTGGGACACTTTTGTGGATCTCT-3′ (SEQ ID NO: 4). Bcl-2-Fwd-5′-TGGGATGCCTTTGTGGAACT-3′ SEQ ID No 2, Bcl-2-Rev-5′-GAGACAGCCAGGAGAAATCAAAC-3′ SEQ ID No 3. Relative expression was determined using the comparative Cₜ method.

DCs were incubated with a titrating dose of APRIL trimer (Zymogenetics) or APRIL/BAFF heterotrimer (Zymogenetics) for 30 min at 37° C. 50 μg/mL of Heparin Sulfate Proteoglycans (HSP) (Sigma) were added to the DCs 30 minutes prior to the addition of APRIL. Membrane-bound APRIL was detected by anti-human APRIL Ab (Alexis Biochemicals).

Next, 10⁵ DCs were plated in a 100 μL volume and stimulated for 24 hours with and without 20 ng/mL e. coli LPS (InvivoGen, CA). IL-10 was measured on a LUMINEX® bead-based platform using a Bio Plex 200 and the Bio Plex Manager 5.0 software (Bio Rad, CA). This system was also utilized to determine the concentration of IL-10 in DC-B cell co-culture systems. P values were acquired by Student t-test using GRAPHPAD PRISM® 5 (GraphPad Software, Inc, CA).

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. As used herein, the phrase “consisting essentially of” limits the scope of a claim to the specified materials or steps and those that do not materially affect the basic and novel characteristic(s) of the claimed invention. As used herein, the phrase “consisting of excludes any element, step, or ingredient not specified in the claim except for, e.g., impurities ordinarily associated with the element or limitation.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a Word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. A method for diagnosing systemic lupus erythematosus (SLE) comprising the steps of:
determining a syndecan-1 expression level of a sample;
comparing the syndecan-1 expression level in the sample to a normal level of expression in a control sample of a known normal tissue; and
correlating the syndecan-1 expression level to a diagnoses of systemic lupus erythematosus, wherein an increased syndecan-1 expression level indicates a diagnoses of systemic lupus erythematosus.

2. The method of claim 1, wherein the sample comprises a tissue sample, a fluid or a supernatant.

3. The method of claim 1, wherein the sample comprises one or more cells from a patient.

4. The method of claim 1, wherein the step of determining the syndecan-1 expression level comprises hybridization with an allele specific probe, an antibody probe, or immunohistochemistry.

5. The method of claim 1, wherein the syndecan-1 expression level is determined by performing mass spectrometry analysis of syndecan-1 nucleic acids obtained from the individual, rolling circle amplification of a portion of a syndecan-1 nucleic acid obtained from the individual, hybridization with an allele specific probe, performing FISH analysis of syndecan-1 nucleic acids obtained from the individual, performing RT-PCR analysis of syndecan-1 nucleic acids obtained from the individual, performing sequencing analysis of syndecan-1 nucleic acids obtained from the individual, hybridization with an antibody probe or immunohistochemistry.

6. The method of claim 1, wherein the control sample is the syndecan-1 expression level obtained at an earlier timepoint.

7. A method for monitoring the progression of systemic lupus erythematosus comprising the steps of:
providing a sample from a patient having systemic lupus erythematosus;
determining a syndecan-1 expression level in the sample;
comparing the syndecan-1 expression level in the sample to a normal level of expression in a control sample of a known normal tissue to determine a SLE progression of the systemic lupus erythematosus; and
evaluating a treatment based on the SLE progression, wherein a decreased syndecan-1 expression level indicates a regression of systemic lupus erythematosus.

8. The method of claim 7, wherein the SLE control sample is obtained at an earlier time point from the patient.

9. The method of claim 7, further comprising the step of recording an initial syndecan-1 expression level as the SLE control sample.

10. The method of claim 6, wherein the step of determining the subject syndecan-1 expression level comprises hybridization with an allele specific probe, an antibody probe, or immunohistochemistry.

11. The method of claim 6, wherein the syndecan-1 expression level comprises performing mass spectrometry analysis of syndecan-1 nucleic acids obtained from the individual, rolling circle amplification of a portion of a syndecan-1 nucleic acid obtained from the individual, hybridization with an allele specific probe, performing FISH analysis of syndecan-1 nucleic acids obtained from the individual, performing RT-PCR analysis of syndecan-1 nucleic acids obtained from the individual, performing sequencing analysis of syndecan-1 nucleic acids obtained from the individual, hybridization with an antibody probe or immunohistochemistry.

12. A method for treating a patient at risk for systemic lupus erythematosus (SLE) comprising the steps of:
obtaining a sample from a patient at risk for systemic lupus erythematosus;
determining a syndecan-1 expression level in the sample;
comparing the syndecan-1 expression level in the sample to a normal level of expression in a control sample of a known normal tissue to determine a SLE progression of the systemic lupus erythematosus;
correlating the syndecan-1 expression level to a diagnoses of systemic lupus erythematosus;
administering an effective amount of a pharmaceutical agent to the patient;
obtaining a second sample from a patient;
determining a second syndecan-1 expression level in the second sample; and
predicting the efficacy of the pharmaceutical agent based on the second syndecan-1 expression level, wherein a decrease in the expression of syndecan-1 indicates a predisposition to responsiveness to the pharmaceutical agent.

13. The method of claim 12, wherein the syndecan-1 expression level is determined by hybridization with an allele specific probe, an antibody probe, or immunohistochemistry.

14. The method of claim 12, wherein the syndecan-1 expression level is determined by performing mass spectrometry analysis of syndecan-1 nucleic acids obtained from the individual, rolling circle amplification of a portion of a syndecan-1 nucleic acid obtained from the individual, hybridization with an allele specific probe, performing FISH analysis of syndecan-1 nucleic acids obtained from the individual, performing RT-PCR analysis of syndecan-1 nucleic acids obtained from the individual, performing sequencing analysis of syndecan-1 nucleic acids obtained from the individual, hybridization with an antibody probe or immunohistochemistry.

15. A method for stratifying a patient in a subgroup of a clinical trial of a lupus erythematosus therapy comprising the steps of:
obtaining a sample from a patient suspected of having lupus erythematosus;
determining a of syndecan-1 expression level in the sample to a normal level of expression in a control sample of a known normal tissue;
administering a candidate drug to the patient;
obtaining a second sample from a patient;
determining a second syndecan-1 expression level in the second sample;
comparing the syndecan-1 expression level to the second syndecan-1 expression level to predict the efficacy of the pharmaceutical agent, wherein a decrease of the syndecan-1 expression level indicates a predisposition to responsiveness to the pharmaceutical agent; and
stratifying the patient into a subgroup for a clinical trial.

16. The method of claim 15, wherein the sample comprises a tissue sample, a fluid or a supernatant.

17. The method of claim 15, wherein the sample comprises one or more cells.

18. The method of claim 15, wherein the syndecan-1 expression level is determined by hybridization with an allele specific probe, an antibody probe, or immunohistochemistry.

19. A method of performing a clinical trial to evaluate a candidate drug believed to be useful in treating a disease state associated with syndecan-1 gene expression, the method comprising:
a) administering a candidate drug to a first subset of patients having lupus erythematosus;
b) administering a placebo to a second subset of the patients having lupus erythematosus;
c) obtaining a sample from the members of the first subset and the second subset;
d) measuring a syndecan-1 expression level from the sample;
e) comparing the syndecan-1 expression level in the sample to a normal level of expression in a control sample of a known normal tissue;
f) determining if there is a statistically significant reduction in the expression of syndecan-1, wherein a statistically significant reduction indicates that the candidate drug is useful in treating said disease state; and
repeating step a) to f).

20. The method of claim 16, wherein the syndecan-1 expression level is measured by hybridization with an allele specific probe, an antibody probe, or immunohistochemistry.

21. A method for modulating systemic lupus erythematosus (SLE) through mediation of a B cell response comprising the steps of:
identifying a subject suspected of having SLE; and
mediating a B cell response by reducing a BAFF level in the subject and reducing a APRIL level in the subject.

22. A method for treating systemic lupus erythematosus (SLE) through mediation of a B cell response comprising the steps of:
providing one or more anti-BAFF antibodies to reduce a BAFF level in the subject;
providing one or more anti-IL-10 antibodies to reduce a BAFF level in the subject; and
providing one or more TACI-Fc compositions to reduce a APRIL level in the subject.

23. A method for mediated an IgG response in a subject comprising the steps of:
identifying a subject in need of treatment;
providing one or more anti-BAFF antibodies; and
providing one or more anti-IL-10 antibodies, wherein the IgG level is reduced.

24. A method for mediated an IgA and an IgM response in a subject comprising the steps of:
identifying a subject in need of treatment; and
providing one or more TACI-Fc, wherein the IgA and the IgM levels are reduced.

25. A method for mediated an IgA response in a subject comprising the steps of:
providing one or more BCMA-Fc compounds to decreased an IgA level without significantly altering an IgM level.

26. A method for reducing auto-antibodies comprising the steps of:
identifying a subject in need of reducing auto-antibodies;
providing one or more anti-BAFF antibodies and/or one or more anti-IL-10 antibodies to reduce a BAFF level in the subject; and
providing one or more TACI-Fc compositions to reduce a APRIL level in the subject.


SEQUENCE LISTING (4 sequences)

SEQ ID NO: 1 - DNA, 18 bp, artificial sequence
  synthetic oligonucleotide
aagcgctcctggcctttc

SEQ ID NO: 2 - DNA, 20 bp, artificial sequence
  synthetic oligonucleotide
tgggatgcctttgtggaact

SEQ ID NO: 3 - DNA, 23 bp, artificial sequence
  synthetic oligonucleotide
gagacagccaggagaaatcaaac

SEQ ID NO: 4 - DNA, 23 bp, artificial sequence
  synthetic oligonucleotide
ctgggacacttttgtggatctct